(19) United States
(12) Patent Application Publication
(10) Pub. No.: US 2004/0186042 A1
(43) Pub. Date: Sep. 23, 2004

(54) Iso-$g(b)-bisabolol as fragrance and aroma substance

(76) Inventors: Gerhard Schmaus, Hoexter-Bosseborn (DE); Manfred Meier, Fuerstenberg (DE); Norbert A Braun, Holzminden (DE); Bernd Hoelscher, Halle (DE); Wilhelm Pickenhagen, Chavannes des Bois (CH)

Correspondence Address: Stephan A Pendorf, Pendorf & Cutliff, 5111 Memorial Highway, Tampa, FL, 33634-7356, US

(21) Appl. No.: 10/483,276

(22) PCT Filed: Jul. 6, 2002
(86) PCT No.: PCT/EP02/07546

(30) Foreign Application Priority Data
Jul. 9, 2001 (DE) 101 32 520.7
Aug. 9, 2001 (DE) 101 39 290.7

(51) Int. Cl. A61K 7/46

(52) U.S. Cl. 512/8

(57) ABSTRACT

The compound (1S/R)-1-[(1S/R)-1,5-dimethyl-hex-5-enyl]-4-methyl-cyclohex-3-en-1-ol (iso-β-bisabolol) of the formula A:

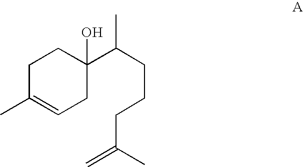

is described, as well as the individual configuration isomers thereof. Iso-β-bisabolol has a very strong flowery and extremely pleasant odor reminiscent of lily-of-the-valley, so that even small amounts of this substance are able to effect a modification of a perfume or flavor (base) composition that can be detected by sensory means.

### FIELD OF THE INVENTION

The invention relates to the novel substance iso-β-bisabolol, i.e. (1S/R)-1-[(1S/R)-1,5-dimethyl-hex-5-enyl]-4-methyl-cyclohex-3-en-1-ol and its use as perfume and/or flavoring agent. The invention furthermore relates to preferred configuration isomers (diastereomers, enantiomers) of iso-β-bisabolol with particularly pronounced sensory properties, as well as corresponding mixtures of configuration isomers.

The invention furthermore relates to perfume and/or flavoring agent compositions that contain an organoleptically active (effective) amount of iso-β-bisabolol or one or more configuration isomers of iso-β-bisabolol.

The invention also relates to methods for modifying a perfume or flavor composition and methods for imparting a perfume or flavor to, or intensifying a perfume or flavor of, a base composition using iso-β-bisabolol (or the corresponding configuration isomers).

And finally the invention also relates to methods for obtaining or preparing iso-β-bisabolol, its configuration isomers and corresponding mixtures of configuration isomers.

### BACKGROUND OF THE INVENTION

In perfuming and flavoring practice there is generally an ongoing requirement for synthetic perfumes and flavoring agents that can be prepared inexpensively and with constant quality, remain stable on prolonged storage, if possible also in contact with other substances, and have desired olfactory or taste characteristics. Perfumes should have pleasant perfume notes of adequate intensity that are as natural as possible and be able to have an advantageous effect on the perfume of cosmetics or industrial consumer goods. Flavoring agents should be easily digestible, be reminiscent of typical flavor components of popular foods or even identical to the latter and be able to make a positive contribution to the flavor of foods, medicaments to be administered orally, and the like. Discovering perfume and flavoring agents that comply with these requirements has proved to be relatively laborious and demands regular extensive research, in particular if the aim is to find interesting novel perfume notes or flavor trends.

The search for suitable perfume or flavoring agents is made more difficult for the person skilled in the art, in particular because of the following factual issues:

The mechanisms of perfume and flavor perception are not known.

Objective quantitative characterisation of a perfume or flavor is not possible.

The relationships between perfume and/or flavor perception, on the one hand, and the chemical structure of the perfume and/or flavoring agent, on the other hand, have not been adequately researched.

Frequently even minor changes in the structural composition of known perfume or flavoring agents give rise to substantial changes in the olfactory or flavor characteristics and lead to an impairment of compatibility for the human organism.

The success of the search for suitable perfume or flavoring agents therefore frequently depends on the intuition of the searcher.

### DETAILED DESCRIPTION

The objective on which the present invention is based is, taking into account the general framework conditions described above, to indicate a perfume and flavoring agent which, in particular, is able to impart a flowery perfume reminiscent of lily-of-the-valley to conventional perfume or flavor base compositions or to modify the existing perfume of such compositions in an advantageous manner.

The substances to be indicated should offer the perfumerer or flavoring specialist an alternative to the perfume agents with a flowery perfume used or described hitherto which can be widely used when composing perfumes or flavors. Specifically, in the creative process of composition, a laborious process, which as a rule is carried out only by specialists, it is not sufficient to use, in the manner of a template, an arbitrary perfume or flavoring agent to which a specific fragrance or flavor aspect has been assigned in the literature in order to obtain a perfume or flavor image that already exists in the imagination. Specifically, the fragrance or flavor characteristic of a composition cannot be precisely predicted in the sense of an addition if only the constituents of the composition are known, since these constituents are subject to unforeseeable interactions in the mixture. The compatibility of a perfume or flavoring agent with the other constituents of a composition and the presence or lack of accompanying aspects that can be detected by the senses and also have an effect on the overall character of the finished composition, without them perhaps being particularly acknowledged in the description of the fragrance of the pure substance, is therefore also important.

The invention is based on the surprising finding that the novel substance iso-β-bisabolol according to formula A below and its configuration isomers according to formulae 1-4 below are outstandingly suitable for use as a perfume and flavoring agent and to achieve the objectives indicated above. Iso-β-bisabolol has a very strong flowery odor that is reminiscent of lily-of-the-valley and is extremely pleasant, so that even small amounts of this substance are able to effect a modification of a perfume or flavor (base) composition that can be detected by the senses.

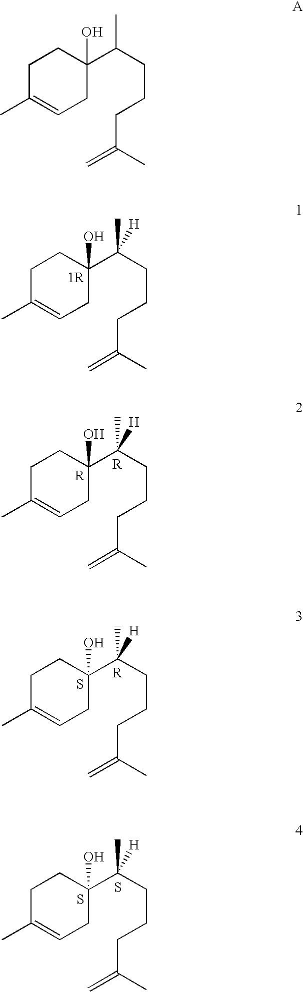

The configuration isomers of iso-β-bisabolol with the R configuration at C1 (carrying a hydroxy functional group) (formulae 1 and 2) are particularly effective from the sensory standpoint. Accordingly, mixtures of two or more configuration isomers of iso-β-bisabolol in which the molar ratio of configuration isomers with the R configuration at C1 to configuration isomers with the S configuration at C1 is greater than 1 and preferably greater than 2 are preferred.

(R,R)-iso-β-bisabolol, d.h. (1R)-1-[(1R)-1,5-dimethyl-hex-5-enyl]-4-methyl-cyclohex-3-en-1-ol is the configuration isomer that is most valuable from the sensory standpoint.

Individual configuration isomers and also mixtures of two or three configuration isomers of iso-β-bisabolol can be obtained from a mixture which contains all configuration isomers of iso-β-bisabolol by conventional separation operations using chiral aids. For example, such a mixture or a fraction of such a mixture can be separated by means of chiral gas chromatography, in which case, in particular, a separation of the configuration isomers R-configured at the C1 atom (particularly valuable from the sensory standpoint) from the corresponding S-configured isomers is then possible; in this context see the examples further below.

Perfume and/or flavoring agent compositions according to the invention contain an organoleptically active (effective) amount of iso-β-bisabolol, it being possible to use both an individual configuration isomer and also a mixture of several configuration isomers of iso-β-bisabolol. Once again, configuration isomers with the R configuration at the C1 atom, in particular (R,R)-iso-β-bisabolol, are preferably used. However, the S-configured configuration isomers can also be used in a corresponding manner.

Existing perfume or flavor compositions can be modified, that is to say changed in respect of their sensory properties, by the addition of an amount of iso-β-bisabolol (with the abovementioned preferences with regard to the configuration isomers) that modify the perfume or the flavor. In particular, existing perfume or flavor compositions can acquire a flowery odor reminiscent of lily-of-the-valley by the addition of iso-β-bisabolol.

To impart a perfume or flavor to, or to intensify a perfume or flavor of, a base composition, which, for example, has no perfume or only a weak perfume in the absence of iso-β-bisabolol, an amount of iso-β-bisabolol (once again with the abovementioned preferences with regard to the configuration isomers) that is effective from the sensory standpoint and the constituents of the base composition can be mixed.

Iso-β-bisabolol is a naturally occurring substance which, for example, can be obtained from East Indian sandalwood oil. However, this was not known hitherto and it is the result of our own research work that on GC-Sniffing (Column: DBWax) of East Indian sandalwood a compound was discovered that was unknown from the sensory standpoint and that it was possible subsequently to identify as iso-β-bisabolol (in this context see the examples further below). East Indian sandalwood oil, that is prepared from the hardwood Santalum album L. (Santalaceae) by steam distillation, is known as one of the oldest perfume raw materials (cf. Lit.: E. J. Brunke, K.-G. Fahlbusch, G. Schmaus, J. Vollhardt, The chemistry of Sandalwood Odor—A Review of the last 10 years. In Revista Italiana EPPOS (Actes des 15èmes Journées Internationales, Huilles Essentielles; Digne-les-Bains, France, Sep. 5-7, 1996) 1997 pp. 49-83). It is also known that the facet-rich, longlasting and difficult to imitate odor of sandalwood oil is to be ascribed not only to the main components but also to the trace components that have a strong odor (cf. Lit.: E.-J. Brunke, G. Schmaus, Dragoco Rep. 1995, 245-257, G. Frater, J. A. Bajgrowicz, P. Kraft, Tetrahedron 1998, 54, 7633-7703). However, in view of the already very extensive work on sandalwood oil, it is to be regarded as extremely surprising that it has now been possible to find a further trace component of sandalwood oil which must be ranked as extremely valuable from the sensory standpoint.

Iso-β-bisabolol can also be obtained if 3,7,11-trimethyl-1,6,10-dodecatrien-3-ol [CAS No. 7212-44-4] (nerolidol) and/or 3,7,11-trimethyl-1,6,11-dodecatrien-3-ol [CAS No. 22143-53-5] (isonerolidol) is cyclized in the presence of acid in such a way that iso-β-bisabolol is formed, optionally alongside other cyclisation products.

The invention will be explained in more detail below on the basis of examples. These relate in particular to methods for obtaining and preparing iso-β-bisabolol as well as methods for separation of the four configuration isomers of iso-β-bisabolol.

### EXAMPLE 1

### Synthesis of (1S/R)-1-[(1S/R)-1,5-dimethyl-hex-5-enyl]-4-methyl-cyclohex-3-en-1-ol, i.e. iso-β-bisabolol

Preliminary Remarks:

According to the method indicated below, the Li Grignard reaction of 6-bromo-2-methyl-hept-1-ene [CAS No.: 38334-97-3; formula 5] with 4-methyl-cyclohex-3-en-1-one [CAS No.: 5259-65-4; formula 6] yields iso-β-bisabolol, i.e. (1S/R)-1-[(1S/R)-1,5-dimethyl-hex-5-enyl]-4-methyl-cyclohex-3-en-1-ol (formula A) in a yield of approximately 3%.

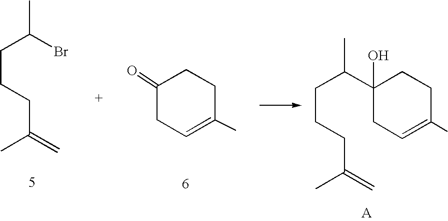

Experimental:

Li powder (0.14 g, 20.2 mmol) is initially introduced into THF (10 ml) under an argon atmosphere at −10° C. 6-bromo-2-methyl-hept-1-ene [CAS No.: 38334-97-3] (1.91 g, 10 mmol) is added slowly dropwise to this mixture and the resulting mixture is stirred for 30 min at −10C. 4-methyl-cyclohex-3-en-1-one [CAS No.: 5259-65-4] (0.88 g, 8 mmol) in THF (5 ml) is then added dropwise and the reaction mixture is stirred for 30 min at −10° C., slowly warmed to room temperature and stirred for a further 8 h at room temperature. Hydrolysis is carried out by slow addition of saturated NH₄Cl solution (10 ml), the organic phase is separated off, the H₂O phase is extracted with Et₂O (3×10 ml) and the combined organic phase is dried over Na₂SO₄. The desiccant is filtered off, the solvent is distilled off in a rotary evaporator and the residue is purified by means of column chromatography (silica gel, hexane/Et₂O 100/0→85:15 in 1% steps, then 80:20, 75:25) and subsequent preparative thin layer chromatography (silica gel, hexane/Et₂O=8:2). Yield: 46.7 mg (2.6%) colorless oil.

Analytical data:

(1S/R)-1-[(1S/R)-1,5-dimethyl-hex-5-enyl]-4-methyl-cyclohex-3-en-1-ol (2 diastereomers): ¹H: 0.91, 0.95 (d, J=6.9 Hz, each 3H), 1.02 (m, 1H), 1.33 (m, 1H), 1.36 (s, 1H, OH), 1.46 (m, 1H), 1.53-1.65 (m, 4H), 1.69 (s, 3H), 1.71 (s, 3H), 1.80-2.07 (m, 4H), 2.12-2.24 (m, 2H), 4.67 (m, 1H), 4.69 (m, 1H), 5.30 (m, 1H). ¹³C: 13.62, 13.66 (q), 22.32, 22.34 (q), 23.23, 23.25 (q), 26.16, 26.29 (t), 26.96, 27.00 (t), 30.33 (t), 30.62, 30.91 (t), 34.18, 34.80 (t), 38.03 (t), 42.05, 42.41 (d), 72.16, 72.18 (s), 109.67, 109.70 (t), 118.38, 118.40 (d), 133.83, 133.91 (s), 146.00,146.04 (s). GC-FTIR (cm⁻¹): μ=3629, 3079, 2972, 2932, 1646, 1449, 1379, 889. GC-MS [m/z (%)]: 222 (4) [M]⁺, 207 (1), 204 (8), 189 (1), 161 (5), 154 (10), 153 (9), 140 (22), 139 (24), 126 (5), 121 (23), 119 (31), 111 (100), 93 (67), 83 (50), 82 (93), 72 (50), 69 (68), 55(57), 43 (34), 41 (35).

### EXAMPLE 2

### Synthesis of (1S/R)-1-[(1S/R)-1,5-dimethyl-hex-5-enyl]-4-methyl-cyclohex-3-en-1-ol, i.e. iso-β-bisabolol

Preliminary Remarks:

The Cu Grignard reaction of 4-bromo-2-methyl-but-1-ene [CAS No.: 20038-12-4] with 2-[(E/Z)-4-methylcyclohex-3-en-1-ylidene]propyl acetate [CAS No.: 90498-67-2; formula 7] yields the prochiral triene 4-[(E/Z)-1,5-dimethylhex-5-enyliden]-1-methylcyclohex-1-ene (formula 8), which is converted to iso-β-bisabolol, i.e. (1S/R)-1-[(1S/R)-1,5-dimethyl-hex-5-enyl]-4-methyl-cyclohex-3-en-1-ol (formula A) by regioselective epoxidation and subsequent reductive epoxide opening.

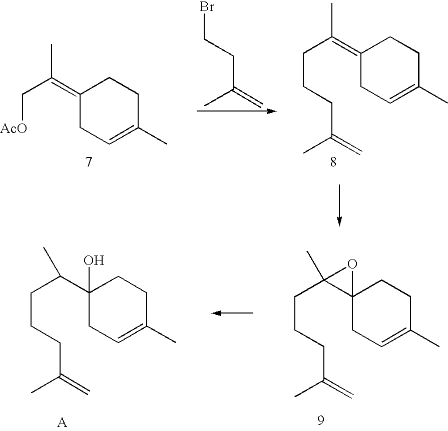

The triene (formula 8; 2 diastereomers=E/Z) is a prochiral intermediate. After separation of the diastereomers (=separation of the E/Z isomers of the triene) racemic iso-β-bisabolol can be prepared as the pure diastereomers by the abovementioned route (epoxidation, reductive expoxide opening) Enantiomer selective epoxidation (e.g. Lit.: W. Zhang, E. N. Jacobsen: “Asymmetric Olefin Epoxidations with Sodium Hypochlorite Catalyzed by Easily Prepared Chiral Mn(III) Salen Complexes.” J. Org. Chem., 1991, 56, 2296-2298) of the pure triene (E- or Z compound) then leads to iso-β-bisabolol in the form of the pure enantiomer (formulae 1-4).

Experimental:

First step: synthesis of 4-(E/Z)-1,5-dimethylhex-5-enylidene]-1-methylcyclohex-1-ene (formula 8):

The Grignard compound is prepared under standard conditions: 4-bromo-2-methyl-but-1-ene [CAS No.: 20038-12-4] (2.0 g, 13.4 mmol) dissolved in THF (5 ml) is added slowly dropwise to Mg shavings (0.40 g, 16.5 mmol) in Et₂O (10 ml); turbidity indicates the initiation of the reaction. The reaction mixture is heated for a further 30 min under reflux and then cooled to −10° C. A 0.1 M solution of Li₂[CuCl₄] (5 ml, 0.5 mmol) in THF is added dropwise and the reaction mixture is stirred for a further 30 min. at −10° C. 2-[(E/Z)-4-methylcyclohex-3-en-1-ylidene]propyl acetate [CAS No.: 90498-67-2; formula 7] (1.94 g, 10 mmol) dissolved in THF (5 ml) is then added dropwise and the reaction mixture is stirred for 2 h at −10° C. and allowed to warm slowly to room temperature. The reaction mixture is cooled to 0° C. and H₂O (10 ml) is added carefully dropwise and the mixture is acidified with 1 M H₂SO₄ (15 ml). The organic phase is separated off and the H₂O phase is extracted with Et₂O (2×40 ml). The combined organic phases are washed with saturated NaCl solution (10 ml) and dried over Na₂SO₄. The desiccant is filtered off, the solvent is distilled off in a rotary evaporator and the residue is purified by column chromatography (silica gel, hexane). Yield: 0.84 g (41%).

GC-MS [m/z (%)]: diastereomer a): 204 (39), 189 (9), 161 (18), 148 (12), 147 (17), 134 (38), 133 (49), 121 (85), 119 (100), 109 (10), 107 (33), 106 (29), 105 (57), 93 (75), 91 (60), 79 (39), 77 (32), 55 (19), 41 (23). diastereomer b): 204 (27), 189 (10), 161 (14), 148 (28), 147 (12), 134 (23), 133 (52), 121 (100), 119 (94), 109 (16), 107 (40), 106 (42), 105 (64), 93 (91), 91 (70), 79 (47), 77 (38), 55 (27), 41 (32).

Second step: synthesis of 2,6-dimethyl-2-(4-methylpent-4-enyl)-1-oxaspiro[2.5]oct-5-ene (formula 9):

A solution of 4-[(E/Z)-1,5-dimethylhex-5-enylidene]-1-methylcyclohex-1-ene (0.25 g, 1.23 mmol) in CH₂Cl₂ (10 ml) is cooled to 0° C., 70% m-CPBA (0.33 g, 1.33 mmol) is then added and the reaction mixture is allowed to warm slowly to room temperature and is stirred for a further 8 h. The solid is filtered off and washed with CH₂Cl₂ (20 ml). The combined organic phases are washed with 10% NaHSO₃ (10 ml), saturated K₂CO₃ solution (10 ml) and NaCl (10 ml) and dried over Na₂SO₄. Yield: 0.22 g (81%).

GC-MS [m/z (%)]: diastereomer a): 220 (2), 205 (6), 202 (4), 187 (3), 159 (3), 152 (5), 151 (4), 147 (5), 138 (10), 137 (14), 132 (8), 119 (10), 111 (14), 110 (43), 109 (17), 107 (7), 105 (10), 95 (100), 94 (25), 93 (28), 91 (22), 81 (17), 79 (63), 77 (24), 69 (14), 68 (16), 67 (22), 55 (23), 43 (19), 41 (19) diastereomer b): 220 (1), 205 (4), 202 (4), 187 (2), 159 (3), 152 (6), 147 (5), 138 (9), 137 (13), 132 (7), 119 (9), 111 (16), 110 (47), 109 (16), 107 (6), 105 (9), 95 (100), 94 (25), 93 (27), 91 (19), 81 (18), 79 (64), 77 (22), 69 (14), 68 (16), 67 (22), 55 (24), 43 (20), 41 (21).

Third step: synthesis of 1-(1,5-dimethylhex-5-enyl)-4-methylcyclohex-3-ene (=iso-β-bisabolol; formula A):

LiAlH₄ (0.1 g, 2.6 mmol) is added carefully to a solution of 2,6-dimethyl-2-(4-methylpent-4-enyl)-1-oxaspiro[2.5]oct-5-ene (0.22 g, 1 mmol) in anhydrous THF (10 ml) and the reaction mixture is heated for 2 h under reflux. H₂O (2 ml) is added carefully dropwise, with cooling; the reaction mixture is then filtered through Theorit 0 (Seitz-Filter-Werke, Bad Kreuznach, Germany) and the residue is washed with Et₂O (40 ml) and dried over Na₂SO₄. The desiccant is filtered off, the solvent is distilled off in a rotary evaporator and the residue is purified by column chromatography (silica gel, hexane/Et₂O=100/0→85:15 in 1% steps, then 80:20, 75:25). Yield: 0.15 g (63%) iso-β-bisabolol as a colorless oil.

Analytical data: see Example 1

### EXAMPLE 3

### Synthesis of (1S/R)-1-[(1S/R)-1,5-dimethyl-hex-5-enyl]-4-methyl-cyclohex-3-en-1-ol, i.e. iso-β-bisabolol

Preliminary Remarks:

The Cu Grignard reaction of 4-bromo-2-methyl-but-1-ene [CAS No.: 20038-12-4] with 1-[2-(4-methylphenyl)sulphonyloxy-1-methylethyl]-4-methylcyclohex-3-en-1-ol tosylate (formula 12 below) also leads to iso-β-bisabolol {=(1S/R)-1-[(1S/R)-1,5-dimethyl-hex-5-enyl]-4-methyl-cyclohex-3-en-1-ol} of the formula A in a yield of approximately 3%.

The tosylate to be used is synthesised in a two-step process [I) regioselective hydroboration of 1-isopropenyl-4-methylcyclohex-3-en-1-ol [CAS No.: 3419-O₂-1, 28342-82-7, 38630-70-5, 73069-45-1] and 11) subsequent tosylation of the primary hydroxyl group].

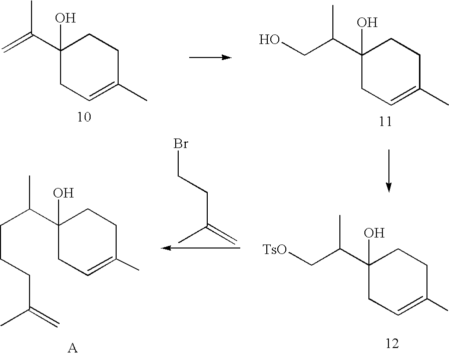

Experimental:

First step: synthesis of 1-(2-hydroxy-1-methylethyl)-4-methylcyclohex-3-en-1-ol (formula 11):

A 0.5 M 9-BBN solution in THF (44 ml, 22 mmol) is added dropwise at 0° C. to a solution of 1-isopropenyl-4-methylcyclohex-3-en-1-ol [CAS No.: 3419-02-1, 28342-82-7, 38630-70-5, 73069-45-1; formula 10] (3.04 g, 20 mmol) in THF (15 ml). The reaction mixture is stirred for 1 h at 0° C. and then at 6 h at room temperature. The solution is cooled to 0° C. and then first H₂O (6 ml) is added and then 3 M NaOH solution (7.3 ml, 22 mmol) and then 30% H₂O₂ solution (7.3 ml, 64.4 mmol) are added dropwise. The reaction mixture is allowed to warm to room temperature and is stirred for a further 3 h. The organic phase is separated off and the H₂O phase is extracted with Et₂O (2×30 ml). The combined organic phases are washed successively with H₂O (2×20 ml), aqueous NaHSO₃ solution (20 ml) and NaCl solution (20 ml) and dried over Na₂SO₄. The desiccant is filtered off, the solvent is distilled off in a rotary evaporator and the residue is purified by column chromatography (silica gel, Et₂O/hexane=1/1). Yield: 1.12 g (33%) of colorless, viscous oil (2 diastereomers) and 2.01 g educt. Based on recovered alcohol, a product yield of 97% results.

¹H: 0.89, 0.98 (d, J=7.1 Hz, je 3H), 1.50 (m, 1H), 1.68 (s, 3H), 1.70-1.82 (m, 2H), 1.85-2.33 (m, 4H), 3.68 (m, 1H), 3.74 (dd, J=4.6, 11.0 Hz, 1H) and 3.79 (dd, J=3.8, 11.0 Hz, 1H), 3.93 (s, 1H, OH), 5.28 (m, 1H). ¹³C: 12.04 (q), 22.96, 23.03 (q), 26.59, 26.71 (t), 29.34, 32.40 (t), 32.81, 36.85 (t), 41.13, 41.96 (d), 64.87, 65.08 (t), 72.98, 73.12 (s), 117.77, 117.93 (d), 133.41, 133.52 (s). GC-MS: diastereomer a): 170 (13), 155 (2), 152 (23), 139 (6), 121 (27), 111 (62), 110 (64), 102 (100), 95 (33), 94 (98), 93 (63), 87 (92), 84 (76), 77 (23), 72 (41), 69 (73), 68 (85), 67 (61), 59 (27), 56 (45), 55 (46), 54 (48), 43 (48), 41 (45) diastereomer b): 170 (11), 155 (2), 152 (19), 139 (5), 121 (22), 111 (61), 110 (62), 102 (95), 95 (31), 94 (100), 93 (60), 87 (92), 84 (73), 77 (22), 72 (39), 69 (69), 68 (82), 67 (59), 59 (29), 56 (43), 55 (45), 54 (45), 43 (46), 41 (42).

Second step: synthesis of 1-[2-(4-methylphenyl)sulphonyloxy-1-methylethyl]-4-methylcyclohex-3-en-1-ol (formula 12):

NEt₃ (2 ml) is added dropwise to a solution of 1-(2-hydroxy-1-methylethyl)-4-methylcyclohex-3-en-1-ol (0.51 g, 3 mmol), p-toluenesulphonic acid (0.63 g, 3.3 mmol) and DMAP (catalytic amount) in CH₂Cl₂ (10 ml), whilst cooling with ice. The reaction mixture is stirred for 12 h at room temperature and H₂O (10 ml) is then added. The organic phase is separated off and the H₂O phase is extracted with CH₂Cl₂ (2×10 ml). The combined organic phases are washed with saturated NaCl solution (10 ml) and dried over Na₂SO₄. The desiccant is filtered off, the solvent is distilled off in a rotary evaporator and the residue is purified by column chromatography (silica gel, Et₂O/hexane=2/1). Yield: 0.79 g (81%) of yellow oil (2 diastereomers).

¹H: 0.93 (d, J=6.9 Hz, 3H) and 0.96 (d, J=7.0 Hz, 3H), 1.41-1.58 (m, 2H), 1.62 (s, 3H), 1.81-1.88 (m, 3H), 2.03-2.15 (m, 2H), 2.18 (s, 1H, OH), 2.42 (s, 3H), 3.93 (dd, J=1.6, 9.5 Hz, 1H) and 3.96 (dd, J=1.5, 9.4 Hz, 1H), 4.27 (dd, J=4.0, 9.5 Hz, 1H) and 4.30 (dd, J=3.9, 9.4 Hz, 1H), 5.19 (m, 1H), 7.33 (d, J=8.1 Hz, 2H), 7.76 (d, J=8.1 Hz, 2H). ¹³C: 11.33, 11.39 (q), 21.13 (q), 22.73, 22.76 (q), 26.31, 26.37 (t), 30.21, 31.41 (t), 33.78, 35.74 (t), 40.79, 41.37 (d), 70.25, 70.39 (s), 72.26, 72.34 (t), 117.26, 117.40 (d), 127.38 (d), 129.44 (d), 132.50 (s), 133.15, 133.25 (s), 144.34 (s).

Third step: synthesis of 1-(1,5-dimethylhex-5-enyl)-4-methylcyclohex-3-en-1-ol (=iso-β-bisabolol; formula A):

The Grignard compound is prepared under standard conditions: 4-bromo-2-methyl-but-1-ene [CAS No.: 20038-12-4] (0.75 g, 5 mmol) dissolved in Et₂O (5 ml) is added slowly dropwise to Mg shavings (0.15 g, 6.2 mmol) in Et₂O (10 ml); turbidity indicates the initiation of the reaction. The reaction mixture is heated for a further 30 min under reflux and then cooled to −20° C. A 0.1 M solution of Li₂[CuCl₄] (2 ml, 0.2 mmol) in THF is added dropwise and the reaction mixture is stirred for a further 30 min at −20° C. 1-[2-(4-methylphenyl)sulphonyloxy-1-methylethyl]-4-methylcyclohex-3-en-1-ol (0.65 g, 2 mmol) dissolved in Et₂O (5 ml) is then added dropwise and the reaction mixture is stirred for 2 h at −20° C. and then allowed to warm slowly to room temperature. The reaction mixture is cooled to 0° C. and H₂O (5 ml) and then saturated NH₄Cl solution (10 ml) are carefully added dropwise. The organic phase is separated off and the H₂O phase is extracted with Et₂O (2×30 ml). The combined organic phases are washed with saturated NaCl solution (10 ml) and dried over Na₂SO₄. The desiccant is filtered off, the solvent is distilled off in a rotary evaporator and the residue is purified by column chromatography (silica gel, hexane/Et₂O 100/0→85:15 in 1% steps, then 80:20, 75:25) and subsequent preparative thin layer chromatography (silica gel, hexane/Et₂O=8:2). Yield: 13.4 mg (3%), colorless oil.

Analytical data: see Example 1

### EXAMPLE 4

### Isolation of Iso-β-bisabolol from East Indian Sandalwood Oil (Santalum Album)

Preliminary Remarks:

On GC sniffing (Column: DBWax) of East Indian sandalwood oil a compound in very low concentration with a strongly flowery, very pleasant odor reminiscent of lily-of-the-valley is discernible by sensory means. This compound is iso-β-bisabolol (formula A). It can be localised by means of GC sniffing and then isolated.

Experimental:

Iso-β-bisabolol is first enriched from East Indian sandalwood by fractional distillation in a Sulzer column. A fraction selection then takes place by means of GC sniffing (as is also the case for the subsequent isolation steps). The fraction from the Sulzer distillation that is strongest from the sensory standpoint is selected by means of GC sniffing and separated off by means of column chromatography (silica gel, step gradient hexane/Et₂O, 1% steps of 100:=to 85:15). The fractions that are strongest from the sensory standpoint are combined and further separated by means of column chromatography (silica gel with 15% AgNO₃, hexane/Et₂0=80:20). The fractions that are strongest from the sensory standpoint are combined again. Finally, the isolation of iso-β-bisabolol takes place by means of two-dimensional preparative capillary gas chromatography [Agilent HP6890; temperature programme 120° C. with 4° C./min to 220° C.; GC column I) DB1 (15 m×0.53 mm×1.2 μm), II) DBWax (27 m×0.53 mm×2 μm); carrier gas He (5.4 ml/min); Detector FID]

Analytical data: see Example 1

### EXAMPLE 5

### Synthesis of (1S/R)-1-[(1S/R)-1,5-dimethyl-hex-5-enyl]-4-methyl-cyclohex-3-en-1-ol, i.e. iso-β-bisabolol, and its Isolation from the Product Mixture

Preliminary Remarks:

The formic acid-catalysed cyclisation of technical grade (E/Z)-nerolidol (=3,7,11-trimethyl-1,6,10-dodecatrien-3-ol) [CAS No. 7212-44-4] (Source: BASF, Cat. No. 203146178) with subsequent saponification (NaOH solution) yields a crude mixture that is subjected to fractional distillation. (Lit.: C. D. Gutsche, J. R. Maycock, C. T. Chang, Tetrahedron 1968, 24, 859-876). On fractional distillation, a fraction is obtained that from the sensory standpoint has a strongly flowery, very pleasant odor reminiscent of lily-of-the-valley, which is to be ascribed to iso-β-bisabolol (structure A). Iso-β-bisabolol can be localised by means of GC sniffing and then isolated.

Experimental:

On GC sniffing (Column: DBWax) of fractions from the acid-catalysed cyclisation of technical grade nerolidol (see above) [I) formic acid, II) sodium hydroxide solution, III) fractional distillation] one fraction is characterised from the sensory standpoint by the presence of a compound with a strongly flowery, very pleasant odor reminiscent of lily-of-the-valley. This compound is iso-β-bisabolol. This is further enriched by renewed fractional distillation in a Sulzer column. Fraction selection here is carried out by sensory means (GC sniffing). The fraction that is strongest from the sensory standpoint on the Sulzer distillation is then separated by means of column chromatography (silica gel, step gradient hexane/Et₂O, 1% steps from 100:0 to 85:15). Fraction selection is again likewise carried out by sensory means (GC sniffing) insofar as no peak is discernible in the chromatogram. The fractions are further purified by means of column chromatography (silica gel with 15% AgNO₃, hexane/Et₂O=80:20). Iso-β-bisabolol can then be detected by means of GC/MS. Finally, isolation of the pure substance is effected by means of two-dimensional preparative capillary gas chromatography [Agilent HP6890; temperature programme 120° C. with 4° C./min to 220° C.; GC column I) DBI (15 m×0.53 mm×1.2 μm), II) DBWax (27 m×0.53 mm×2 μm); carrier gas He (5.4 ml/min); Detector flame ionisation detector.]

### EXAMPLE 6

### Chiral Separation of the Configuration Isomers of Iso-β-bisabolol

Separation of iso-β-bisabolol from Example 2 (2 diastomeric enantiomer pairs, i.e. 4 configuration isomers in total) is effected via chiral gas chromatography. The gas chromatography conditions were as follows:

| | |
|---|---|
| Equipment | Agilent GC6890, Gerstel KAS 4 |
| Sample | 0.2% solution in hexane |
| Injection | 1 μl in the split (split ratio 1:20) |
| Temperature program | 80° C. with 1° C. /min to 150° C. |
| GC column Column | 25 m × 0.25 mm × 0.15 μm) Ivadex-3 |
| material | (IVA analytical technique) |
| | a. 2,3-di-O-acetyl-6-O-tert-butyl-dimethylsilyl-β- |
| | cyclodextrin (30%), PS 086 (70%) |
| Carrier gas | N₂ (1.4 ml/min) |
| Detection | GC sniffing, flame ionisation detector |
| | (split ratio 1:1) |

Separation by means of chiral gas chromatography yields 3 fractions (peaks; ratio=2:1:1), which are designated by A, B, C in Table 1 below. GC sniffing shows that peak A has a very strong flowery, very pleasant odor reminiscent of lily-of-the-valley; peaks B and C, on the other hand, have only a weak odor.

TABLE 1

| Peak | Compound according to formula | Configuration |
|---|---|---|
| A | 1 and 2 | (R,S), (R,R) |
| B | 3 | (S,R) |
| C | 4 | (S,S) |

### EXAMPLE 7

### Seperation of the Configuration Isomers of Iso-β-bisabolol

Seperation of the diastereomer pairs according to formulae 1 and 3 (first pair) and 2 and 4 (second pair), respectively, is first carried out starting from the synthetic mixture of configuration isomers according to Example 2 by means of preparative HPLC (silica gel).

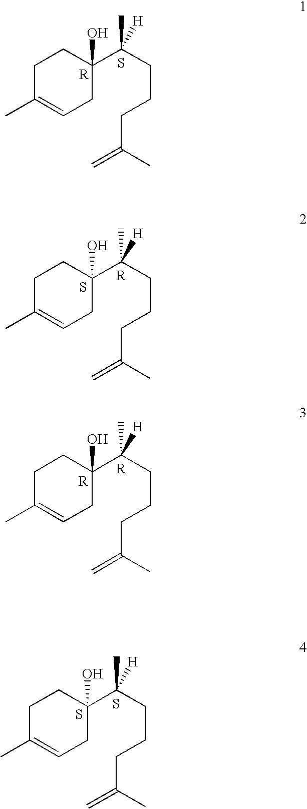

Suitable HPLC condition are:

| | |
|---|---|
| Equipment | Knauer HPLC Pump 64 |
| Sample | Synthetic mixture of diastereomers (1:1:1:1) |
| Injection | 150 μl 25% solution in the solvent |
| Solvent | Hexane/diethyl ether = 9:1 |
| HPLC column | Knauer Eurospher 100 Si 5 μm(250 × 20 mm) |
| Flow | 10 ml/min |
| Detector | Knauer UV Detector |
| Detection wavelength | λ = 220 nm |

The separation yields 2 fractions in accordance with Table 2 below:

TABLE 2

| | Compound | |
|---|---|---|
| | according to | |
| Fraction | formula | Configuration |
| 1 | 2 and 4 | (R,R), (S,S) |
| 2 | 1 and 3 | (R,S), (S,R) |

Subsequent chiral gas chromatography leads to complete separation of the configuration isomers (1/3+2/4).

Under the GC conditions according to Example 6, GC sniffing shows that the compound according to formula 2 possesses a very strong flowery, very pleasant odor reminiscent of lily-of-the-valley, whilst the compounds according to formulae 4, 1, 3 have a weaker odor. The compound according to formula 2 is (R,R)-iso-β-bisabolol, i.e. (1R)-1-[(1R)-1,5-dimethyl-hex-5-enyl]-4-methyl-cyclohex-3-en-1-ol.

What is claimed is:

1. (1S/R)-1-[(1S/R)-1,5-dimethyl-hex-5-enyl]-4-methyl-cyclohex-3-en-1-ol (iso-β-bisabolol) of the formula A:

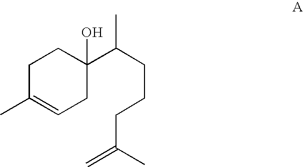

2. Configuration isomer or mixture of configuration isomers of iso-β-bisabolol, selected from the group that consists of (1R)-1-[(1R)-1,5-dimethyl-hex-5-enyl]-4-methyl-cyclohex-3-en-1-ol, (1R)-1-[(1S)-1,5-dimethyl-hex-5-enyl]-4-methyl-cyclohex-3-en-1-ol and mixtures thereof.

3. Mixture of two or more configuration isomers of iso-β-bisabolol, wherein the molar ratio of the configuration isomer or configuration isomers with the R configuration at C1 to the configuration isomer or configuration isomers with the S configuration at C1 and/or the molar ratio of (1R)-1-[(1/R)-1,5-dimethyl-hex-5-enyl]-4-methyl-cyclohex-3-en-1-ol to (1R)-1-[(1/S)-1,5-dimethyl-hex-5-enyl]-4-methyl-cyclohex-3-en-1-ol is greater than 1, preferably greater than 2.

4. (a) Configuration isomer or (b) mixture of two or three configuration isomers of iso-β-bisabolol that has a flowery perfume and is obtainable by a method with the following steps:
preparation of a mixture of all configuration isomers of iso-β-bisabolol
separation of the mixture, or of a fraction separated off therefrom, by means of chiral gas chromatography.

5. Perfume and/or flavoring agent composition comprising an organoleptically active amount of iso-β-bisabolol, a configuration isomer of iso-β-bisabolol or a mixture of two or more configuration isomers of iso-β-bisabolol as perfume and/or flavoring agent.

6. Use of iso-β-bisabolol, a configuration isomer of iso-β-bisabolol or a mixture of two or more configuration isomers of iso-β-bisabolol as perfume and/or flavoring agent.

7. Method for modifying a perfume or flavor composition, wherein an amount of iso-β-bisabolol, a configuration isomer of iso-β-bisabolol or a mixture of two or more configuration isomers of iso-β-bisabolol that modifies the perfume or the flavor is added to the perfume or flavor composition.

8. Method for imparting a perfume or flavor to or intensifying a perfume or flavor of a base composition, characterised in that (a) an amount of iso-β-bisabolol, a configuration isomer of iso-β-bisabolol or a mixture of two or more configuration isomers of iso-β-bisabolol that is effective from the sensory standpoint and
(b) constituents of the base composition are mixed.

9. Method for the preparation of iso-β-bisabolol, a configuration isomer of iso-β-bisabolol or a mixture of two or more configuration isomers of iso-β-bisabolol, wherein 2,6-dimethyl-2-(4-methylpent-4-enyl)-1-oxaspiro[2.5]oct-5-ene is converted to iso-β-bisabolol with reductive opening of the epoxy group.

10. Method for obtaining iso-β-bisabolol, in which the latter is isolated from Santalum album.

11. Method for the preparation of iso-β-bisabolol, wherein 3,7,11-trimethyl-1,6,10-dodecatrien-3-ol [CAS No. 7212-44-4] (nerolidol) and/or 3,7,11-trimethyl-1,6,11-dodecatrien-3-ol [CAS No. 22143-53-5] (isonerolidol) is cyclized in the presence of acid in such a way that iso-β-bisabolol is formed, optionally alongside other cyclisation products.

12. Method for obtaining (a) a configuration isomer or (b) a mixture of two or three configuration isomers of iso-β-bisabolol with a flowery perfume, comprising the following steps:
preparation of a mixture of all configuration isomers of iso-β-bisabolol
separation of the mixture, or of a fraction of the mixture separated off therefrom, by means of chiral chromatography.

13. Method for the preparation (a) of a pure configuration isomer of iso-β-bisabolol or (b) of a mixture of two configuration isomers of iso-β-bisabolol that are enantiomers with respect to one another, in a ratio of at least 2:1, wherein one or more pure enantiomer or enantiomer-enriched precursors are used for selective generation of the chiral centres.
